(19) United States
(12) Patent Application Publication
(10) Pub. No.: US 2024/0261104 A1
(43) Pub. Date: Aug. 8, 2024

(54) DEVICES AND METHODS RELATED TO PULMONARY WAVE MODIFICATION

(71) Applicant: EDWARDS LIFESCIENCES CORPORATION, Irvine, CA (US)

(72) Inventors: Zachary Christopher Berwick, Tustin, CA (US); Allen Jeong Keel, Irvine, CA (US); Yaeer E. Lev, Torrance, CA (US)

(21) Appl. No.: 18/638,351

(22) Filed: Apr. 17, 2024

Related U.S. Application Data
(63) Continuation of application No. PCT/US2022/047458, filed on Oct. 21, 2022.
(60) Provisional application No. 63/271,898, filed on Oct. 26, 2021.

(51) Int. Cl. A61F 2/24; A61F 2/00; A61F 2/04

(52) U.S. Cl. CPC A61F 2/2476; A61F 2002/0081; A61F 2002/043; A61F 2230/0067; A61F 2230/0091

(57) ABSTRACT

According to certain aspects, an implant device comprises an outer member configured to position the implant device within one or more pulmonary vessels. The implant device also comprises an inner member comprising one or more blades configured to modify one or more of forward waves or backward waves within the one or more pulmonary vessels to reduce interference between the forward waves and the backward waves during systole, the one or more blades having a shape or surface that directs a portion of the forward waves to a region of the one or more pulmonary vessels.

### RELATED APPLICATION

This application is a continuation of International Patent Application No. PCT/US2022/047458, filed Oct. 21, 2022 and entitled DEVICES AND METHODS RELATED TO PULMONARY WAVE MODIFICATION, which claims priority to U.S. Provisional Patent Application No. 63/271,898, filed on Oct. 26, 2021 and entitled DEVICES AND METHODS RELATED TO PULMONARY WAVE MODIFICATION, the complete disclosures of which are hereby incorporated by reference in their entireties.

### BACKGROUND

### Field

The present disclosure generally relates to the field of medical devices and procedures.

### Description of Related Art

Implant devices can be positioned at various sites in the human body to regulate fluid flow. For example, implant devices can be used in one or more vessels to provide control of or affect the flow of blood. Artificial control of blood flow with implant devices within one or more vessels can be used to treat various conditions, including pulmonary hypertension.

### SUMMARY

Described herein are one or more devices and/or methods to facilitate pulmonary wave modification. In some implementations, the present disclosure relates to an implant device comprising an outer member configured to position the implant device within one or more pulmonary vessels, and an inner member comprising one or more blades configured to modify one or more of forward waves or backward waves within the one or more pulmonary vessels to reduce interference between the forward waves and the backward waves during systole.

In some implementations, the one or more blades can have a shape or surface that directs a portion of the forward waves to a region of the one or more pulmonary vessels. In some instances, the one or more blades are configured to divert the forward waves to a region of the one or more pulmonary vessels. The one or more blades can flare outward to divert the forward waves.

In some examples, the one or more blades include a curved blade configured to converge the backward waves. The curved blade can include a plurality of fenestrations to allow at least some of the forward waves to pass through. The curved blade may divert at least some of the forward waves to a region of the one or more pulmonary vessels.

In some implementations, the one or more blades have a helical configuration to create a helical flow. The helical configuration can include a predetermined number of rotations for a diameter length of the inner member. The one or more blades can have a linear shape at a cross section across a diameter of the inner member.

In some instances, the one or more blades include one or more streamlined convex surfaces to accelerate a velocity of the forward waves. The one or more blades can be configured to promote a Bernoulli effect on pulmonary pressure and velocity. The velocity of the forward waves can be higher at a distal end than the velocity of the forward waves at a proximal end. The one or more blades can have a biconvex shape.

In some examples, the one or more blades include a first conical section configured to direct the forward waves to a periphery of the one or more pulmonary vessels, and forward waves returned from the periphery cancel out a portion of the forward waves. The one or more blades can include a second conical section configured to accelerate a velocity of the forward waves. A tapering angle of the first conical section can be different from a tapering angle of the second conical section. The cancellation of the portion of the forward waves can reduce the backward waves.

In some implementations, the implant device is positioned within a main vessel. In certain implementations, the implant device is positioned within a branch vessel. In other implementations, the implant device is positioned within a main vessel and one or more branch vessels.

In some instances, a portion of the implant device in one branch vessel and a portion of the implant device within another branch vessel are asymmetrical. In certain instances, the one or more blades are positioned at a plurality of locations within the one or more pulmonary vessels.

In some examples, the outer member and the inner member are formed as a single continuous tube. In some implementations, the reduction of the interference between the forward waves and the backward waves reduces pulmonary pressure and right ventricular afterload. In some instances, the implant device is a valved device positioned within a branch vessel and configured to prevent the backward waves from reaching a main vessel during systole.

In some aspects, the one or more blades include a ribbed portion along a wall of the inner member that is configured to create friction. The ribbed portion can be configured to accelerate flow of the forward waves through a center of the inner member. In certain aspects, the one or more blades include a spiked portion or a studded portion along a wall of the inner member that is configured to create friction. The spiked portion or the studded portion can be configured to accelerate flow of the forward waves through a center of the inner member.

In some implementations, the one or more blades include a constriction along the inner member that is configured to accelerate flow of the forward waves. In certain implementations, the one or more blades include an asymmetrical choke point that is configured to create asynchrony between the backward waves from a first branch pulmonary vessel and a second branch pulmonary vessel. The first branch pulmonary vessel can be a left pulmonary artery, and the second branch pulmonary vessel can be a right pulmonary artery. In some examples, the asymmetrical choke point is configured to accelerate flow of the forward waves through a center of the inner member. In some aspects, the implant device is sterilized.

In some implementations, the present disclosure relates to a method of delivering an implant device. The method comprises positioning a distal portion of a delivery catheter at a target position within a vessel, the implant device being in a compressed configuration and being within the distal portion of the delivery catheter. The implant device comprises an outer member configured to position the implant device within one or more pulmonary vessels, and an inner member comprising one or more blades configured to modify one or more of forward waves or backward waves within the one or more pulmonary vessels to reduce interference between the forward waves and the backward waves during systole. The method further comprises causing the implant device to assume an expanded configuration after releasing the implant device from the delivery catheter. The method also comprises positioning the implant device in the expanded configuration at a target site. In certain aspects, the one or more blades can have a shape or surface that directs a portion of the forward waves to a region of the one or more pulmonary vessels. The above method(s) can be performed on a living animal or on a simulation, such as on a cadaver, cadaver heart, anthropomorphic ghost, simulator (e.g., with body parts, heart, tissue, etc. being simulated).

In some implementations, the present disclosure relates to an implant device comprising means for positioning the implant device within one or more pulmonary vessels, and means for modifying one or more of forward waves or backward waves within the one or more pulmonary vessels to reduce interference between the forward waves and the backward waves during systole. In certain aspects, the means for modifying can be configured to direct a portion of the forward waves to a region of the one or more pulmonary vessels.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 illustrates an example representation of a human heart in accordance with one or more examples.

FIG. 2A is a view of an outer member of an implant device for modifying pulmonary waves in accordance with one or more examples.

FIG. 2B is a view of an implant device for modifying pulmonary waves in accordance with one or more examples.

FIG. 3A is a view of an inner member of an implant device for modifying pulmonary waves in accordance with one or more examples.

FIG. 3B provides cross-sectional views of an inner member of an implant device for modifying pulmonary waves in accordance with one or more examples.

FIG. 4A is a view of an inner member of an implant device for modifying pulmonary waves in accordance with one or more examples.

FIG. 4B provides cross-sectional views of an inner member of an implant device for modifying pulmonary waves in accordance with one or more examples.

FIG. 5A is a view of a helical flow created by an inner member of an implant device for modifying pulmonary waves in accordance with one or more examples.

FIG. 5B provides cross-sectional views of an inner member of an implant device for modifying pulmonary waves and a heat map associated with the inner member of the implant device in accordance with one or more examples.

FIG. 5C is a perspective view of an inner member of an implant device for modifying pulmonary waves in accordance with one or more examples.

FIG. 5D is a perspective view of an inner member of an implant device for modifying pulmonary waves in accordance with one or more examples.

FIG. 6A is a view of an inner member of an implant device for modifying pulmonary waves in accordance with one or more examples.

FIG. 6B provides cross-sectional views of an inner member of an implant device for modifying pulmonary waves in accordance with one or more examples.

FIG. 6C provides views of implant devices in accordance with one or more examples.

FIG. 7 is a view of an inner member of an implant device for modifying pulmonary waves in accordance with one or more examples.

FIG. 8 is a view of an implant device for modifying pulmonary waves in accordance with one or more examples.

FIG. 9A is a perspective view of an implant device for modifying pulmonary waves in accordance with one or more examples.

FIG. 9B is a perspective view of an implant device for modifying pulmonary waves in accordance with one or more examples.

FIG. 9C is a perspective view of an implant device for modifying pulmonary waves in accordance with one or more examples.

FIG. 10 is a view of an implant device for modifying pulmonary waves in accordance with one or more examples.

FIG. 11 is a perspective view of an implant device for modifying pulmonary waves in accordance with one or more examples.

FIG. 12 provides a flow diagram illustrating a process for implanting an implant device for modifying pulmonary waves in accordance with one or more examples.

### DETAILED DESCRIPTION

The headings provided herein are for convenience only and do not necessarily affect the scope or meaning of the claimed invention.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Certain standard anatomical terms of location are used herein to refer to certain device components/features and to the anatomy of animals, and namely humans, with respect to the preferred examples. Although certain spatially relative terms, such as “proximal,” “distal,” “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

### OVERVIEW

In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

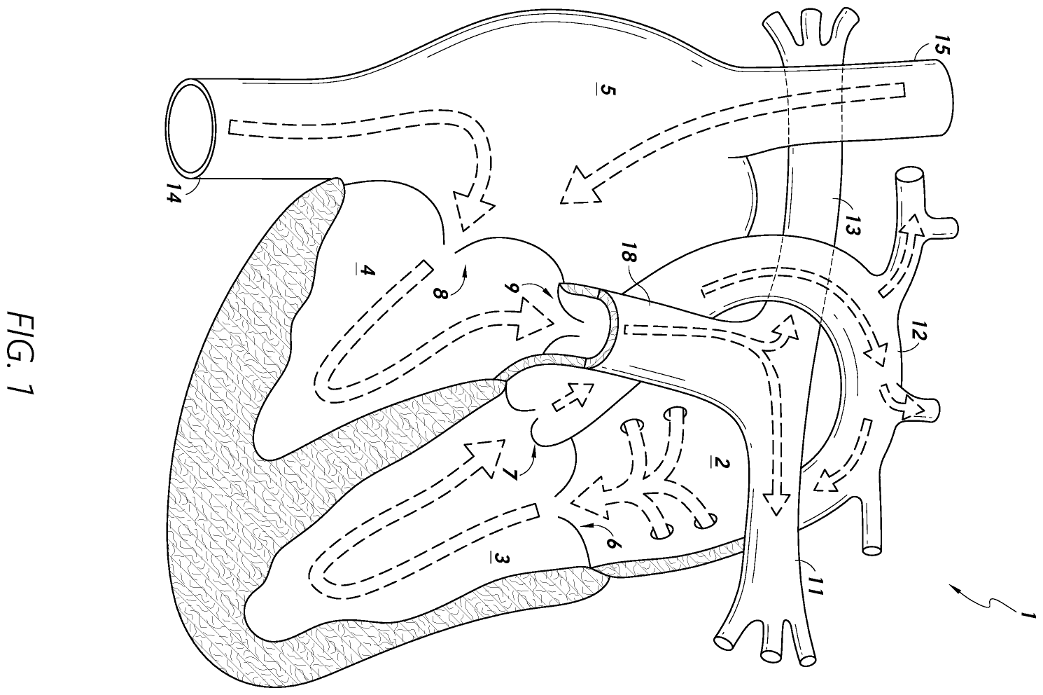

FIG. 1 illustrates an example representation of a heart 1 including indicators for example blood flow. The heart 1 includes four chambers, namely the left atrium 2, the left ventricle 3, the right ventricle 4, and the right atrium 5. A wall of muscle, referred to as the septum, separates the left 2 and right 5 atria and the left 3 and right 4 ventricles.

The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (e.g., systole) and open during ventricular expansion (e.g., diastole). The valves of the heart 1 further include the pulmonary valve 9, which separates the right ventricle 4 from the pulmonary artery 18, and may be configured to open during systole so that blood may be pumped toward the lungs, and close during diastole to prevent blood from leaking back into the heart 1 from the pulmonary artery 18. The pulmonary valve 9 generally has three cusps/leaflets, wherein each one may have a crescent-type shape. The pulmonary artery 18 branches into a right pulmonary artery 13 and a left pulmonary artery 11. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The four valves ensure that blood does not flow in the wrong direction during the cardiac cycle; that is, to ensure that the blood does not back flow through the valve. Blood flows from the venous system (e.g., via the superior vena cava 15 and the inferior vena cava 14) and the right atrium 4 through the tricuspid valve 8 to the right ventricle 4, then from the right ventricle 4 through the pulmonary valve 9 to the pulmonary artery 18 and the lungs. Oxygenated blood then flows through the mitral valve 6 from the left atrium 2 to the left ventricle 3, and finally from the left ventricle 3 through the aortic valve 7 to the aorta/arterial system.

Pulmonary hypertension is a rapidly deteriorating vascular disease associated with high short-term mortality rates. A primary driver of disease progression is the increase in pulmonary arterial pressure due to a reduction in vascular compliance. The reduction in compliance is caused by several key factors, for example, remodeling of the microcirculation or arteriosclerosis due to elevated pressures or systemic inflammation, respectively. Sustained and progressive increases in pulmonary arterial pressure can result in right ventricular-vascular uncoupling whereby the right ventricle 4 is no longer able to compensate for the increase in afterload and eventually fails.

Right ventricular (RV) afterload includes resistive and pulsatile components. Compared to other circulations, the pulsatile component of RV afterload is uniquely large in the pulmonary circulation due to a highly compliant and rapidly branching vasculature. Under normal conditions, pulmonary vessels dilate in response to systolic blood flow, which return to normal diameter during diastole due to elastic recoil. During the diastolic recoil period, a pressure-velocity wave is formed, which is reflected backwards from the distal microcirculation and branch points toward the right ventricle 4. This observation may be referred to as the backward reflection wave and under normal conditions does not impact the RV afterload since it occurs during diastole. However, under conditions of pulmonary hypertension, the ability of vessels to expand in response to increases in pressure-flow is lost due to remodeling and stiffening of the vasculature. Under such conditions, the backward wave is reflected much sooner and with greater magnitude, during the systolic phase of the right ventricle 4. Because the reflection is occurring during contraction, the right ventricle 4 has to generate more force to overcome the increase in fluid impedance, leading to elevated pulsatility which is characteristic of pulmonary hypertension patients.

The present disclosure provides devices (including various medical implants) and methods for modifying pulmonary waves. The term “implant” is used herein according to its plain and/ordinary meaning and may refer to any medical implant, frame, valve, shunt, stent, anchor, and/or similar devices for use in treating various conditions in a human body. Implants may be delivered via catheter (e.g., transcatheter) for various medical procedures and may have a generally sturdy and/or flexible structure. The term “catheter” is used herein according to its broad and/ordinary meaning and may include any tube, sheath, steerable sheath, steerable catheters, and/or any other type of elongate tubular delivery device comprising an inner lumen configured to slidably receive instrumentation, including for example delivery catheters and/or cannulas.

The term “associated with” is used herein according to its broad and/ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

Certain examples are disclosed herein in the context of cardiac implants and procedures. However, although certain principles disclosed herein are particularly applicable to the anatomy of the heart, it should be understood that implant devices and implantation procedures in accordance with the present disclosure may be implanted in, or configured for implantation in, any suitable or desirable anatomy.

### Pulmonary Wave Modification

The present disclosure relates to systems, devices, and methods for pulmonary wave modification. For instance, the present disclosure relates to implant devices and associated implantation methods for modifying pulmonary waves. According to certain aspects, implant devices according to the present disclosure can be configured to modify pulmonary waves, which can include forward waves and backward reflection waves. For example, forward waves and/or backward reflection waves in pulmonary arteries (or pulmonary vessels) can be modified to reduce interference between the forward waves and the backward reflection waves. In some cases, an implant device can include a vessel positioning outer member and a wave altering inner member, which are described in detail below. The outer member can position the implant device within pulmonary arteries. The inner member can modify pulmonary waves, for example, forward waves and/or backward reflection waves. In some implementations, the outer member and the inner member can be separate. In other implementations, the outer member and the inner member can be integrated or formed as a single continuous structure or member. Implant devices according to the present disclosure can reduce the impact of backwards reflection waves to reduce pulsatile loading and therefore RV afterload in order to preserve RV function. For instance, reducing the interference between the forward waves and the backward reflection waves can reduce pulmonary pressure and RV afterload. The implant devices according to the present disclosure may be applicable across multiple types of pulmonary hypertensive conditions. According to certain aspects, backward waves in pulmonary arteries (e.g., flowing toward the pulmonary valve 9) may be referred to as “backward(s) compression waves,” “return reflected waves,” “backward(s) reflected/reflection waves,” or “backward(s) waves.” Forward waves in pulmonary arteries (e.g., flowing away from the pulmonary valve 9) may be referred to as “forward compression waves” or “forward waves.”

Implant devices according to the present disclosure can include various configurations that promote diversion, convergence, deflection, and/or cancellation of pulmonary waves. For example, implant devices can be configured to divert, converge, deflect, and/or cancel forward waves or backward waves or both in order to minimize the interference of the forward waves and the backward waves. For instance, the implant devices can reduce the impedance and fluid energy loss that occur when forward waves and backward waves that were previously in different phases of the cardiac cycle interact due to both now occurring during systole. By mitigating the wave interference, the resistance to forward flow is reduced, thereby decreasing RV work. Pulmonary pressure and RV afterload can be reduced by reducing the interference between the forward waves and the backward waves. Such approach can reduce RV afterload without requiring modification of the pulmonary microcirculation that determines pulmonary vascular resistance, or left atrial pressures. A secondary benefit can be promotion of fluid shear across the luminal surface, which improves vessel reactivity and flow-mediated vasodilation. Various examples and implementations that modify pulmonary waves can be applicable across various circulations and diseases, including but not limited to arterial hypertension and coronary vascular disease. Features described with respect to various examples and implementations of the implant devices according to the present disclosure can be independently implemented.

### Outer Member

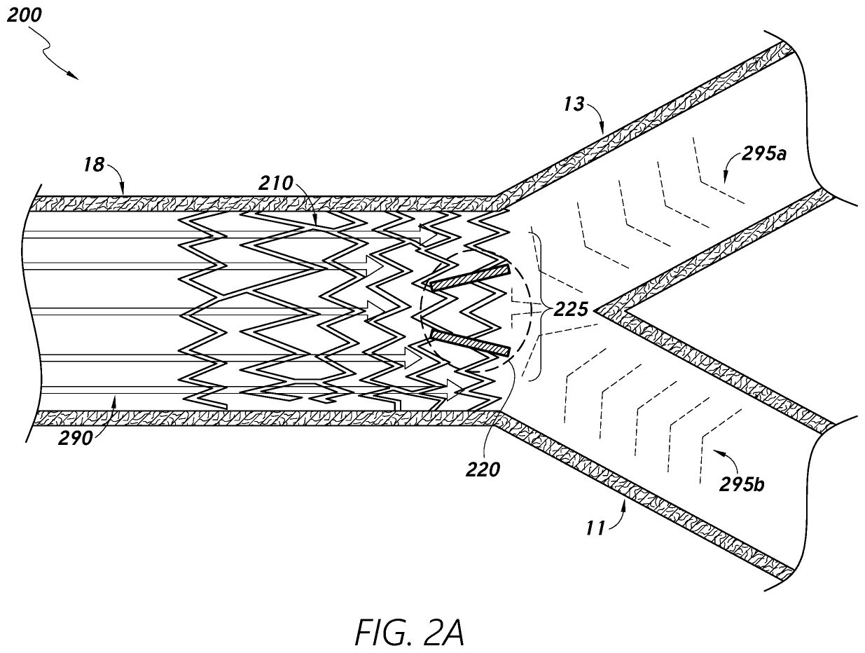

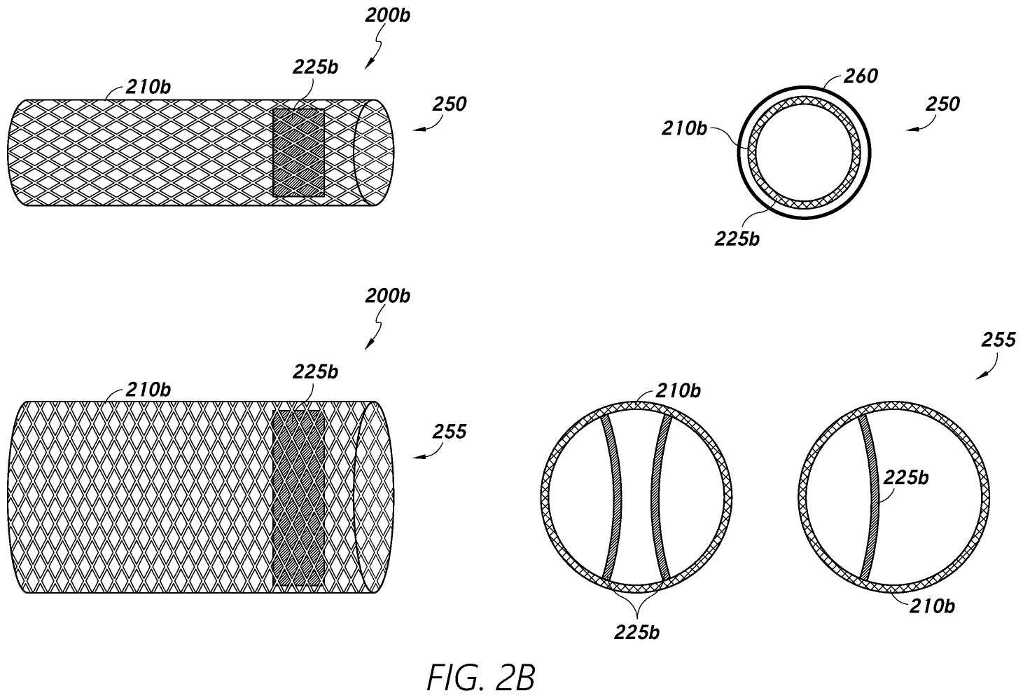

FIG. 2A is a view of an outer member 210 of an implant device 200 for modifying pulmonary waves in accordance with one or more examples. In FIG. 2A, the main pulmonary artery (PA) 18 branches into the right pulmonary artery (RPA) 13 and the left pulmonary artery (LPA) 11. For illustrative purposes, the outer member 210 is shown as being positioned at a location right before the split of the PA 18 into the RPA 13 and the LPA 11, but the outer member 210 can be positioned anywhere within one or more pulmonary arteries as appropriate.

FIG. 2A also shows an inner member 220, which can include one or more blades 225 configured to modify pulmonary waves. Forward waves flow away from the pulmonary valve (not shown), and backward waves flow toward the pulmonary valve. In the example of FIG. 2A, forward waves 290 (shown in solid lines) in the PA 18 flow toward the RPA 13 and the LPA 11. Backward waves 295a (shown in dashed lines) in the RPA 13 flow toward the PA 18, and backward waves 295b (shown in dashed lines) in the LPA 11 flow toward the PA 18. The backward waves 295a and the backward waves 295b may converge where the RPA 13 and the LPA 11 meet. In some cases, the backward waves 295a and 295b may tend to flow along the inner track or the outer track, indicating a location for manipulation of direction due to interference with the forward waves 295a.

The outer member 210 can position the device 200 within one or more pulmonary arteries (PAs) in various configurations. In some implementations, the outer member 210 can be positioned within the main PA 18. In other implementations, the outer member 210 can be positioned within a branch PA, such as the RPA 13 and the LPA 11. In additional implementations, the outer member 210 can originate in the main or parent PA and extend into one or more branch or daughter PAs. The device 200 is described as being placed in one or more PAs for illustrative purposes, but may be also be placed in pulmonary vessels or other vessels as appropriate in order to modify waves or blood flow in such vessels.

The outer member 210 may be positioned at an appropriate target site within one or more pulmonary arteries. In one implementation, the outer member 210 may be positioned closer to the pulmonary valve. In another implementation, the outer member 210 may be positioned closer to the branching of the RPA 13 and the LPA 11. The target site for the outer member 210 can be determined based on various factors, such as remodeling and/or stiffening of pulmonary circulation, pre-implant diagnostic evaluation, wave intensity analysis, etc. For instance, the outer member 210 may be positioned closer to the pulmonary valve if the degree of remodeling and/or stiffening of the PAs is greater. Many variations are possible. In some cases, the target site may be determined based on simulation and/or testing.

In some implementations, the outer member 210 includes a plurality of self-expanding or balloon expandable struts, referred to as a “stent.” In one implementation, as discussed above, the stent is positioned within the main PA 18. In another implementation, the stent is positioned with the branch PAs. In an additional implementation, the stent originates in the main or parent vessel and extends into the branch or daughter vessel. The “branch stenting” may occur within the main PA 18, branch PAs, or lobular PAs.

The outer member 210 can be continuous with an inner member 220, for example, made of a single or multiple raw material extrusions that are fixed via thermal or mechanical methods. The outer member 210 can be made of any suitable material, for example, materials for forming a stent.

In certain implementations, asymmetry is designed in regards to the RPA 13 and LPA 11 in order to minimize the coincidence of the right backward waves 295a versus left backward waves 295b. By creating differences in timing, the afterload seen at the pulmonary valve and thus the right ventricle can be reduced by minimizing the probability of constructive interference of these backward waves. Features described with respect to various examples and implementations of the outer member and/or the inner member can be independently implemented.

### Inner Member

The inner member of an implant device can be configured to modify pulmonary waves, for example, forward waves and/or backward waves. The inner member can include various examples and implementations to promote diversion, convergence, deflection, and/or cancellation of pulmonary waves in order to minimize the interference forward waves and backward waves. According to certain aspects, the inner member can be configured to deflect forward waves toward a selected region of one or more vessels, which can reduce interference with backward waves. The inner member can direct the forward waves away from the backward waves. As an example, the inner member may direct the forward waves to the periphery of the one or more vessels. In the present disclosure, the periphery of one or more vessels is discussed as a selected region to which the forward waves can be directed for illustrative purposes, and any region within the vessel(s) can be selected as appropriate. The inner member can include one or more blades configured to modify the forward waves and/or the backward waves. The term “blade” is used herein according to its plain and/ordinary meaning. A blade can be exposed to flow within a vessel and have any shape or surface configured to change or direct the flow (e.g., forward waves and/or backward waves). Examples of blades can include blades configured to divert flow, converge flow, create helical flow, accelerate flow, cancel waves, etc. Examples of blades may include diverting blades, converging blades, helical blades, Bernoulli blades, blades having streamlined surfaces, convex blades, biconvex blades, conical blades, biconical blades, etc. Many variations are possible.

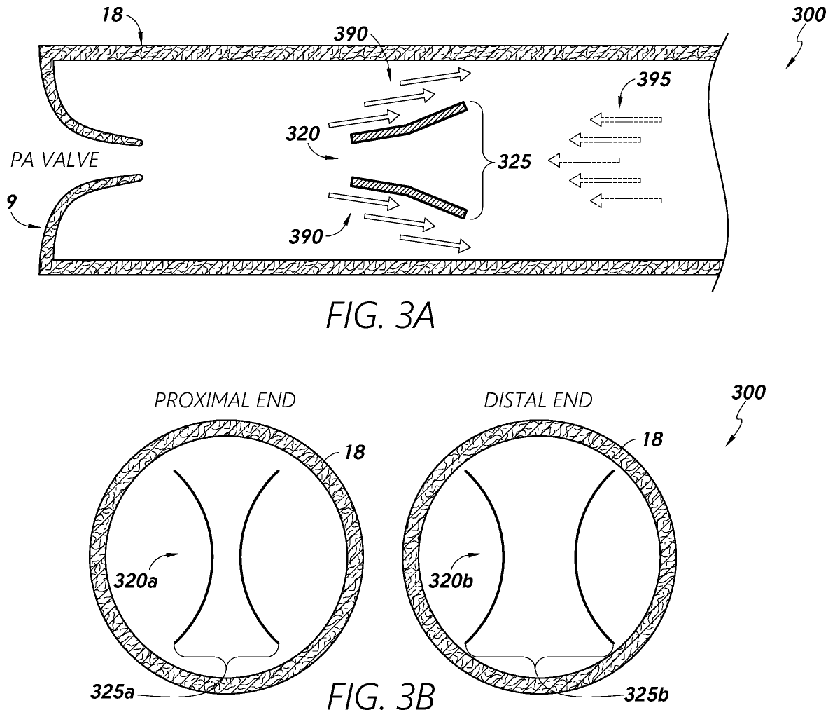

The inner member and the blades may be constructed of any suitable material, such as a mesh or solid material. Examples of materials can include, but are not limited to, Nitinol, polytetrafluoroethylene (PTFE), expanded PTFE (ePTFE), polyetheretherketone (PEEK), other synthetic materials established as safe for intravascular use, textiles, biomaterials such as pericardium, etc. The outer member can also be constructed of any suitable material, including the examples of materials described above. Various examples and implementations of the inner member are described below. In some of FIGS. 3A-11, the outer member is not shown to illustrate the inner member more clearly, but it is understood that the implant device includes an outer member with the inner member. Features described with respect to various examples and implementations of the outer member and/or the inner member can be independently implemented.

In some implementations, as described above, the outer member and the inner member (e.g., the blades) of an implant device can be formed as a single continuous structure or member. For instance, the inner member and/or the blades can be integrated with the outer member or formed from the same material as the outer member (e.g., cut from the same tube). FIG. 2B is a view of an implant device 200b for modifying pulmonary waves in accordance with one or more examples, where the outer member and the inner member are formed as a single continuous structure. In such cases, the outer member of the implant device 200b may refer to the outward facing surface that contacts the wall of a vessel, and the inner member of the implant device 200b may refer to the inward facing surface or portion of the implant device 200b that includes one or more blades configured to modify flow.

FIG. 2B shows an implant device 200b that is formed from a single continuous tube of material in a collapsed/compressed state 250 and an expanded state 255. As an example, the implant device 200b may be made of a single Nitinol tube. The implant device 200b can be delivered in a delivery system, and can initially be in the compressed state 250 and transition to the expanded state 255 for deployment. In the example of FIG. 2B, the implant device 200b is a stent 210b including one or more blades 225b. FIG. 2B includes side views and cross-sectional views of the stent 210b and the blades 225b in the compressed state 250 and the expanded state 255, respectively. In the cross-sectional view of the compressed state 250, the blade 225b is continuous with the outer circumference of the stent 210b. An outer sheath 260 of a delivery catheter is also shown. In the cross-sectional view of the expanded state 255, the blade 225b is expanded to its target dimensions. In some cases, the stent 210b can include dual blades 225b or a single blade 225b (e.g., asymmetrical), as shown in the cross-sectional view. The blade 225b can be continuous with the outer circumference of the stent 210b while collapsed/compressed inside a delivery system. Once deployed, the outer circumference of the stent 210b expands while the blade 225b portion expands to a lesser degree and returns to its nominal shape as determined by geometric constraints and/or shape setting techniques. For example, a portion (e.g., the blade 225b) of the stent 210b can be designed to not self-expand or expand only to the target blade dimension, while the remaining portion of the stent 210b expands to contact the vessel, functioning as anchoring points. In this manner, a complex geometry can be created from a single tube. For instance, the inner member of the implant device 200b can have various configurations as described in FIGS. 3A-11.

### A. Wave Diversion

FIG. 3A is a view of an inner member 320 of an implant device 300 for modifying pulmonary waves in accordance with one or more examples. In some implementations, the inner member 320 is configured for wave diversion. The device 300 is shown to be positioned close to the pulmonary valve 9 within the pulmonary artery 18. The inner member 320 can include a single, double, or plurality of blades 325. The blades 325 can divert forward waves to a selected region (e.g., the periphery) of the vessel away from backward waves. The blades 325 can be designed with the appropriate axial and/or cross-sectional angles to promote forward waves away from the returning incident backward waves to limit interference as described herein. FIG. 3A provides a visual depiction of wave diversion via representative wave profiles, where solid arrows represent forward waves 390 and dashed arrows represent backward waves 395. In the example of FIG. 3A, the blades 325 flare outward toward the periphery of the vessel. The blades 325 in FIG. 3A are provided as an example, and the blades 325 may have any shape or surface that is suitable for diverting the forward waves 390 to the periphery of the vessel. As described above, the blades 325 may be constructed of any suitable material, including a mesh or solid material such as PTFE or other synthetic materials previously established as safe for intravascular use. The wave diverting device 300 can be placed in the main PA 18 and/or within branch PAs as described herein.

FIG. 3B provides cross-sectional views of an inner member 320 of an implant device 300 for modifying pulmonary waves in accordance with one or more examples. For instance, FIG. 3B shows cross-sectional views of the inner member 320 of FIG. 3A at proximal and distal ends. Reference numbers 320a and 325a indicate the inner member 320 and the blades 325 at the proximal end, respectively. Reference numbers 320b and 325b indicate the inner member 320 and the blades 325 at the distal end, respectively. The blades 325a of the inner member 320a at the proximal end are placed closer toward the center of the vessel(s). The blades 325b of the inner member 320b at the distal end are placed farther apart and closer toward the periphery of the vessel(s). Accordingly, the blades 325 of the inner member 320 flare outward from the proximal end to the distal end to divert forward waves 390 to the periphery of the vessel(s). In the example of FIG. 3B, the blades 325a, 325b have a curved shape at the cross section, but the blades 325 can have any shape that is appropriate to divert the forward waves 390 to the periphery of the vessel(s).

As described herein, the target position of the implant device 300 can be determined based on different factors, such as remodeling and/or stiffening of vessels. In addition, one or more blades 325 can be stacked. For instance, sets of blades 325 can be placed at multiple locations along the longitudinal axis of the inner member 320 (e.g., along the length of the vessel). Features relating to examples and implementations described with respect to FIGS. 3A-3B, for example, associated with wave diversion, can be independently implemented from features relating to examples and implementations described with respect to other figures of the present disclosure.

### B. Wave Convergence

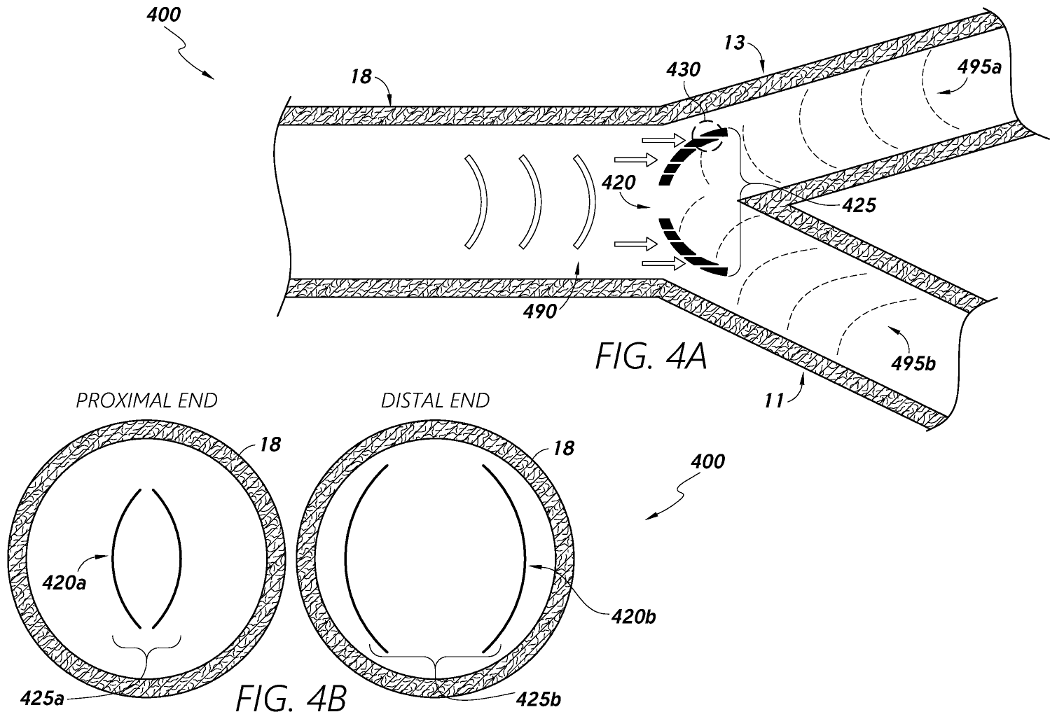

FIG. 4A is a view of an inner member 420 of an implant device 400 for modifying pulmonary waves in accordance with one or more examples. In some implementations, the inner member 420 is configured for wave convergence. The device 400 is shown to be positioned within the pulmonary artery 18 where the PA 18 branches into the RPA 13 and the LPA 11. The inner member 420 can include a single, double, or plurality of blades 425. For instance, the inner member 420 may include one or more curved blades. Forward waves 490, represented by solid lines and arrows, flow within the main PA 18 toward the RPA 13 and the LPA 11. Backward waves 495a, represented by dashed lines, flow within the RPA 13 toward the main PA 18. Backward waves 495b, represented by dashed arrows, flow within the LPA 11 toward the main PA 18. The blades 425 can converge the right backward waves 495a and the left backward waves 495b such that they cancel each other out. The blades 425 can also divert some of the forward waves 490 to a selected region (e.g., the periphery) of the vessel away from the backward waves 495a, 495b, for example, in a manner similar to the example of FIG. 3A.

The blades 425 can have a curved shape to converge the right backward waves 495a and the left backward waves 495b. For example, the blades can be curved on the distal surface and have a concave curvature. The concave curvature can be at a specified angle to promote convergence of the backward waves 495a, 495b. For instance, the blades 425 can be designed with the appropriate axial and/or cross-sectional angles to converge the backward waves 495a, 495b. The angle of the curvature may be determined such that the blades 425 promote collision of the right backward waves 495a and the left backward waves 495b to cancel each other out and prevent flowing further into the pulmonary artery 18. The angle of the curvature may also take into account diversion of the forward waves 490. The inner surface of the blades 425 is reflective such that the right backward waves 495a and the left backward waves 495b reflect from the inner surface and converge to cancel out. The curved shape of the blades 425 can allow the backward waves 495a, 495b to converge more quickly over a shorter distance. In some implementations, the blades 425 can have a straight shape over a distance that is sufficient to converge the backward waves 495a, 495b.

In some cases, having the blades 425 within the pulmonary artery 18 can increase resistance to the forward waves 490. Accordingly, in some implementations, the blades 425 can include one or more fenestrations or openings 430 to allow some of the forward waves 490 to pass through the blades 425, thereby reducing resistance for the forward waves 490. The fenestrations 430 can be at a particular angle that minimizes resistance to the forward waves 490. The blades 425 in FIG. 4A are provided as an example, and the blades 425 may have any shape or surface that is suitable for converging the backward waves 495a, 495b and/or diverting the forward waves 490. As described above, the blades 425 may be constructed of any suitable material, including a mesh or solid material such as PTFE or other synthetic previously established as safe for intravascular use. The wave converging device 400 can be placed in the main PA 18 and/or within branch PAs as described herein.

FIG. 4B provides cross-sectional views of an inner member 420 of an implant device 400 for modifying pulmonary waves in accordance with one or more examples. For instance, FIG. 4B shows cross-sectional views of the inner member 420 of FIG. 4A at proximal and distal ends. Reference numbers 420a and 425a indicate the inner member 420 and the blades 425 at the proximal end, respectively. Reference numbers 420b and 425b indicate the inner member 420 and the blades 425 at the distal end, respectively. The blades 425a of the inner member 420a at the proximal end are placed closer toward the center of the vessel(s). The blades 425b of the inner member 420b at the distal end are placed farther apart and closer toward the periphery of the vessel(s). The blades 425 of the inner member 420 flare outward from the proximal end to the distal end with a concave curvature to converge the backward waves 495a, 495b. The blades 425 of the inner member 420 can also divert the forward waves 490 to the periphery of the vessel(s). In the example of FIG. 4B, the blades 425a, 425b have a curved shape at the cross section, but the blades 425 can have any shape that is appropriate to converge the backward waves 495a, 495b from the RPA 13 and the LPA 11.

As described herein, the target position of the implant device 400 can be determined based on different factors, such as remodeling and/or stiffening of vessels. In addition, one or more blades 425 can be stacked. For instance, sets of blades 425 can be placed at multiple locations along the longitudinal axis of the inner member 420 (e.g., along the length of the vessel). Features relating to examples and implementations described with respect to FIGS. 4A-4B, for example, associated with wave convergence, can be independently implemented from features relating to examples and implementations described with respect to other figures of the present disclosure.

### C. Helical Flow

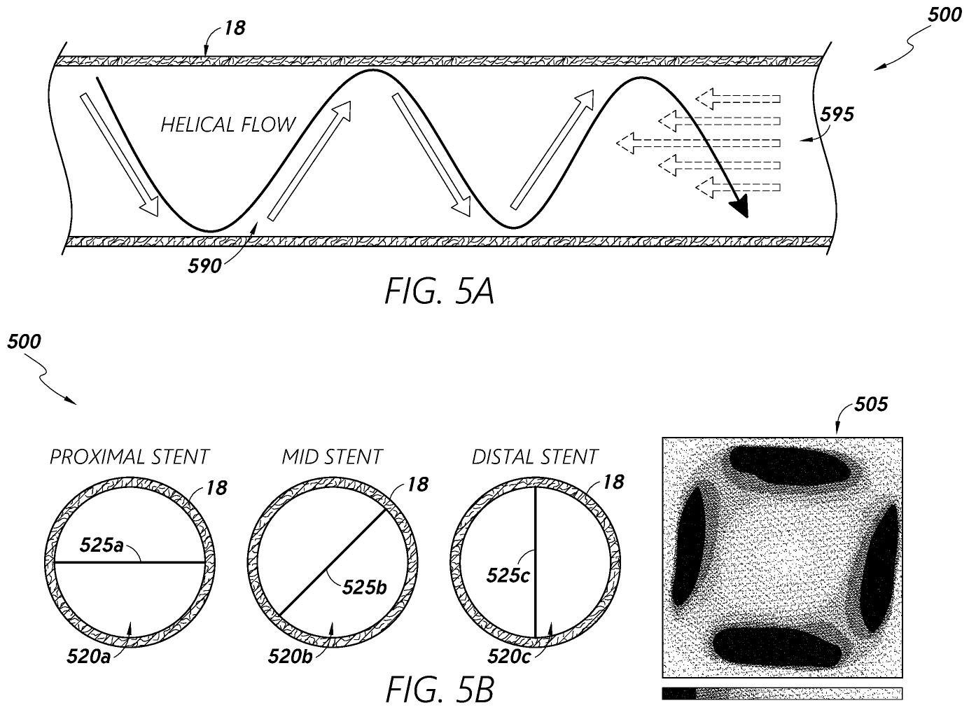

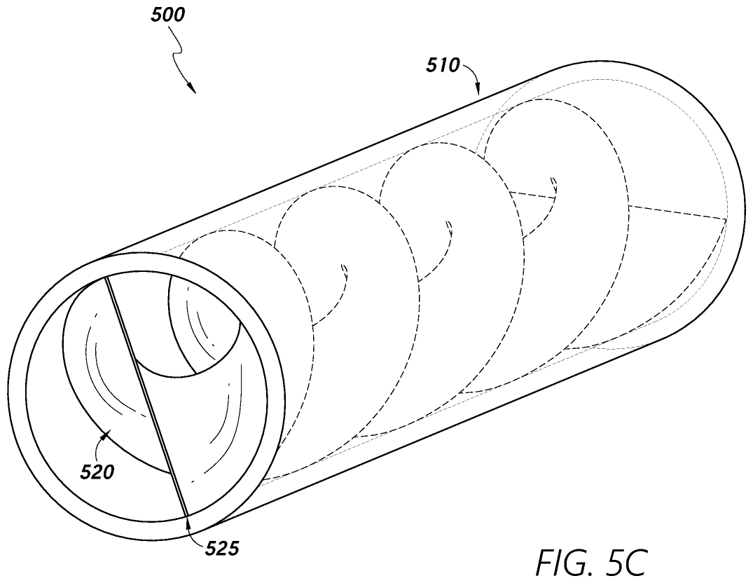

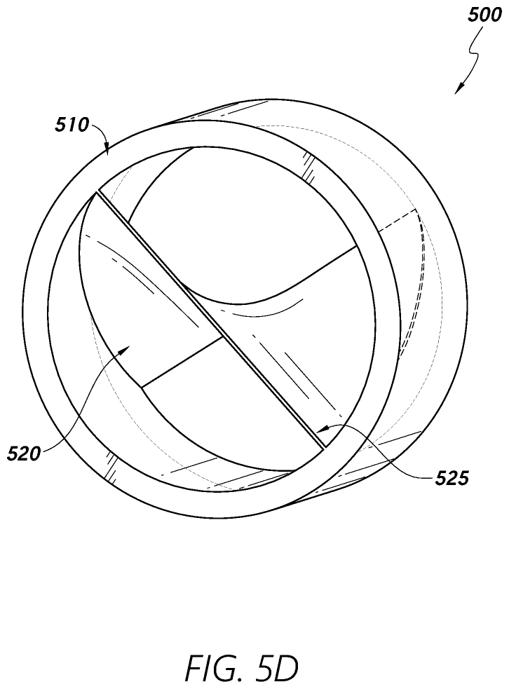

FIGS. 5A-5D provide different views relating to an inner member 520 of an implant device 500 for modifying pulmonary waves in accordance with one or more examples. In some implementations, the inner member 520 is configured for helical flow. The inner member 520 can include a single, double, or plurality of blades 525. The blade 525 can create a helical flow within the vessel(s). The blade 525 can be static, but twist axially moving from the proximal end to the distal end. The presence of the twisting blade 525 promotes a helical flow pattern that accelerates flow into a selected region (e.g., the periphery) of the vessel and away from the incident backward waves. According to certain aspects, the tangential or circumferential flow characteristics can promote fluid shear and vessel reactivity.

FIG. 5A shows an example helical flow that can be created by an implant device 500 (not shown). In the example of FIG. 5A, the implant device 500 can be positioned within the pulmonary artery 18. Forward waves 590, represented by solid arrows, can follow a helical flow and move toward the periphery of the vessel(s) such that the forward waves 590 do not interfere with backward waves 595, represented by dashed arrows. As described above, the blade 525 may be constructed of any suitable material, including a mesh or solid material such as PTFE or other synthetic materials previously established as safe for intravascular use. The helical flow device 500 can be placed in the main PA 18 and/or within branch PAs as described herein.

FIG. 5B provides cross-sectional views of an inner member 520 of an implant device 500 for modifying pulmonary waves in accordance with one or more examples. For instance, FIG. 5B shows cross-sectional views of an inner member 520 of an implant device 500 at proximal, middle, and distal sections. In some implementations, the implant device 500 can be a stent, and the cross sections are denoted as “proximal stent,” “mid stent,” and “distal stent” in FIG. 5B. Reference numbers 520a and 525a indicate the inner member 520 and the blade 525 at the proximal section, respectively. Reference numbers 520b and 525b indicate the inner member 520 and the blade 525 at the middle section, respectively. Reference numbers 520c and 525c indicate the inner member 520 and the blade 525 at the distal section, respectively. In the example of FIG. 5B, a blade 525 in the shape of a straight line spans across the diameter of the inner member 520. In FIG. 5B, the blade 525a, 525b, 525c has a straight or linear shape at the cross section, but the blade 525 can have any shape that is appropriate to create a helical flow for forward waves 590, for example, deflect the forward waves 590 to the periphery of the vessel(s).

FIG. 5B also provides a heat map 505 associated with the inner member 520 of the implant device 500 in accordance with one or more examples. For instance, the heat map 505 can represent computational fluid dynamics (CFD) analysis of fluid velocity. The heat map 505 shows velocity of flow at the cross section of the vessel(s), with a darker shade representing higher velocity and a lighter shade representing lower velocity. It can be seen that the implant device 500 leads to higher velocity at the periphery of the vessel, which represents helical flow.

FIG. 5C is a perspective view of an inner member 520 of an implant device 500 for modifying pulmonary waves in accordance with one or more examples. The implant device 500 includes an outer member 510 and an inner member 520. The inner member 520 includes a blade 525 that twists axially to form a helical structure. In some implementations, the blade 525 can have a predetermined number of turns or rotations. For example, the blade 525 can have a predetermined number of rotations for the length of the diameter of the blade 525 or the inner member 520. The number of rotations of the blade 525 per diameter length can be determined as appropriate, for example, to create a desired helical flow pattern, sufficient fluid spin, etc. In some implementations, the number of rotations of the blade 525 can be up to 2. In other implementations, the number of rotations of the blade 525 can be more than 2. Any suitable number of rotations can be used. In some cases, the blade 525 may be described by a pitch and/or an angle as appropriate.

FIG. 5D is a perspective view of an inner member 520 of an implant device 500 for modifying pulmonary waves in accordance with one or more examples. FIG. 5D is similar to FIG. 5C, but shows a quarter (90°) turn of the blade 525. The blade 525 shown in FIGS. 5B-5D is provided as an example, and the blade 525 may have any helical configuration or other shape that is suitable for creating a helical flow.

As described herein, the target position of the implant device 500 can be determined based on different factors, such as remodeling and/or stiffening of vessels. In addition, one or more blades 525 can be stacked. For instance, sets of blades 525 can be placed at multiple locations along the longitudinal axis of the inner member 520 (e.g., along the length of the vessel). Features relating to examples and implementations described with respect to FIGS. 5A-5D, for example, associated with helical flow, can be independently implemented from features relating to examples and implementations described with respect to other figures of the present disclosure.

### D. Streamlined Flow/Bernoulli Blade

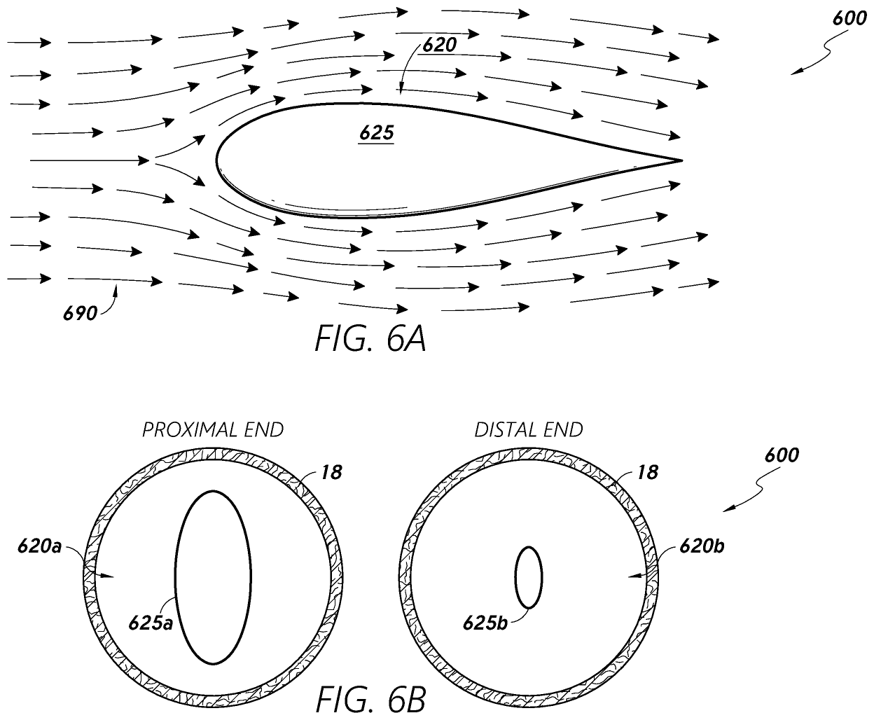

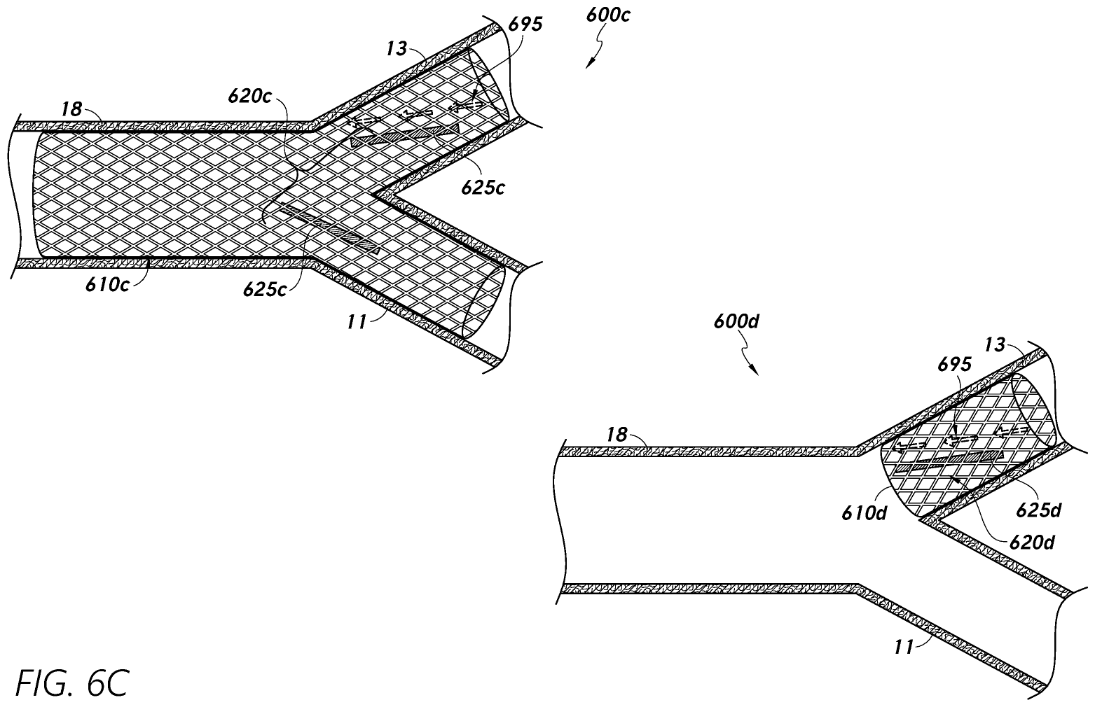

FIG. 6A is a view of an inner member 620 of an implant device 600 for modifying pulmonary waves in accordance with one or more examples. In some implementations, the inner member 620 is configured for streamlined flow. The inner member 620 can include a single, double, or plurality of blades 625. The blades 625 can be configured to accelerate flow velocity, for example, in a manner similar to aerodynamic principles of flight. In the example of FIG. 6A, the blade 625 can have a biconvex shape and can accelerate flow velocity of the forward waves 690. In some cases, the blade 625 having a biconvex shape may be referred to as a “Bernoulli blade.” The velocity of the forward waves 690 can be increased, pushing the forward waves 690 toward a selected region of the vessel(s), such as the periphery. The forward waves 690 can accelerate past backward waves. The biconvex shape may be such that velocity is higher on distal surfaces versus proximal surfaces. In some cases, this can be used to shift the forward waves 690 and the backward waves out of phase, for example, such that they occur during a period of the cardiac cycle that impacts RV afterload.

The blade 625 can be designed with appropriate axial and/or cross-sectional angles to promote acceleration of forward waves 690 past backward waves (not shown) to limit interference between the forward waves 690 and the backward waves. The biconvex shape is shown in FIG. 6A for illustrative purposes, and the blade 625 can have any shape that can accelerate flow velocity of the forward waves 690. For instance, the blade 625 can include one or more convex surfaces or any streamlined surfaces. The blade 625 can include any surface or geometric modification that would promote favorable characteristics of pressure and flow velocity. For example, the blade 625 can be configured to promote a Bernoulli effect on pulmonary pressure and velocity. As described above, the blade 625 may be constructed of any suitable material, including a mesh or solid material such as PTFE or other synthetic materials previously established as safe for intravascular use. The streamlined flow device 600 can be placed in the main PA 18 and/or within branch PAs as described herein.

FIG. 6B provides cross-sectional views of an inner member 620 of an implant device 600 for modifying pulmonary waves in accordance with one or more examples. For instance, FIG. 6B shows cross-sectional views of the inner member 620 of FIG. 6A at proximal and distal ends. Reference numbers 620a and 625a indicate the inner member 620 and the blade 625 at the proximal end, respectively. Reference numbers 620b and 625b indicate the inner member 620 and the blade 625 at the distal end, respectively. The blade 625a of the inner member 620a at the proximal end tapers toward the distal end. Accordingly, the blade 625b of the inner member 620b at the distal end is smaller than the blade 625a of the inner member 620a at the proximal end. In the example of FIG. 6B, the blade 625a, 625b have an elliptical shape at the cross section, but the blade 625 can have any shape that is appropriate to accelerate velocity of forward waves 690, for example, to the periphery of the vessel(s).

In certain implementations, asymmetry can be applied for the implant device 600 in regards to different branch vessels (e.g., RPA 13 and LPA 11). The blade 625 can be placed in a position or a vessel such that backward waves from the branch vessels would not converge at the same magnitude. For instance, in general, backward waves coming from each branch meet and become additive. As an example, if the blade 625 is placed in one of the branches and a part of the main vessel (e.g., the main pulmonary artery), summation of the backward waves from the different branches can be reduced or prevented. If the backward waves from the different branches can be moved out of phase, the overall resistance may be decreased for the subsequent cardiac beat.

FIG. 6C provides views of implant devices 600c, 600d in accordance with one or more examples. Asymmetry of the blade can be provided with respect to different branch vessels in order to promote acceleration of flow in one of the vessels, for example, preferential to the position of backward waves versus forward waves. An implant device may be positioned in both branch vessels or in a single branch vessel. There can be some asymmetry with backward waves. For example, if the backward waves are positioned towards the inner or the outer luminal border, a blade described above, such as a Bernoulli blade, would accelerate the backward waves. For instance, velocity increases as the lumen narrows. By having asymmetry in the branch vessels, for example, by having an asymmetrical stent 610c or a single branch stent 610d, convergence of backward waves 695 from one branch can avoid summation with backward waves 695 from the other branch. As shown in FIG. 6C, an implant device 600c can be an asymmetrical stent 610c positioned in a main vessel (e.g., PA 18) and both branch vessels (e.g., RPA 13 and LPA 11). The stent 610c can include an inner member 620c with a blade 625c for each branch, and the blades 625c can be asymmetrical. Alternatively, as shown in FIG. 6C, an implant device 600d may be a single branch stent 610d placed in one of the branch vessels (e.g., RPA 13 or LPA 11) and include an inner member 620d having a blade 625d.

As described herein, the target position of the implant device 600, 600c, 600d can be determined based on different factors, such as remodeling and/or stiffening of vessels. In addition, one or more blades 625 can be stacked. For instance, sets of blades 625 can be placed at multiple locations along the longitudinal axis of the inner member 620 (e.g., along the length of the vessel). Features relating to examples and implementations described with respect to FIGS. 6A-6C, for example, associated with streamlined flow and/or Bernoulli blade, can be independently implemented from features relating to examples and implementations described with respect to other figures of the present disclosure.

### E. Wave Cancellation

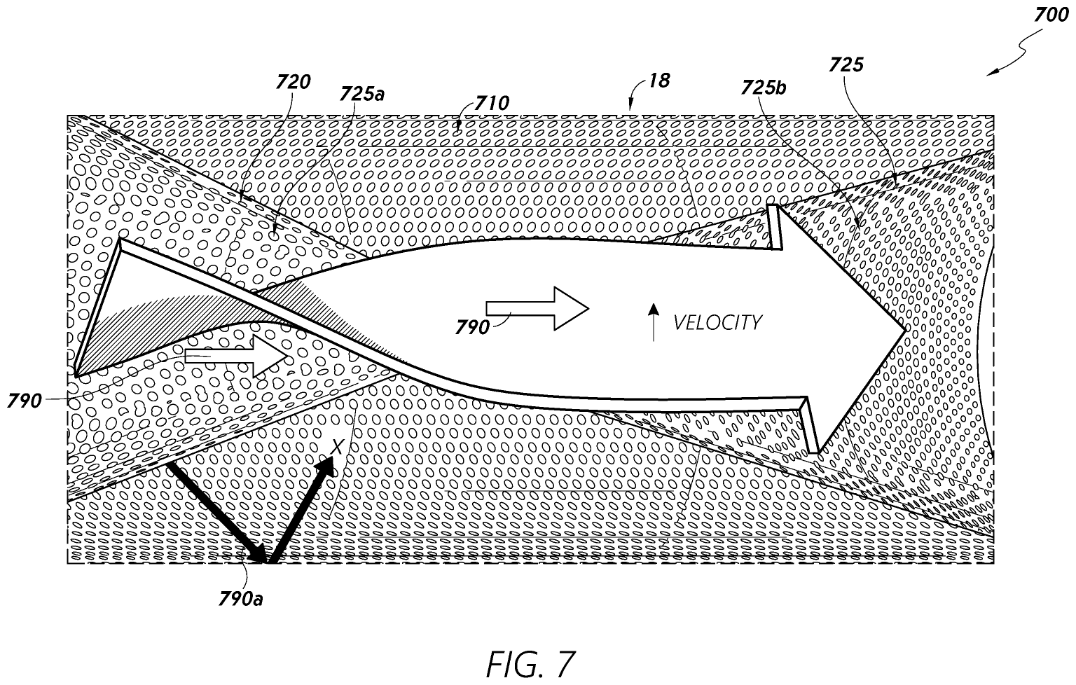

FIG. 7 is a view of an inner member 720 of an implant device 700 for modifying pulmonary waves in accordance with one or more examples. In some implementations, the inner member 720 is configured for wave cancellation. In the example of FIG. 7, the implant device 700 can be positioned within the pulmonary artery 18. The outer member 710 is also shown in FIG. 7. The inner member 720 can include a single, double, or plurality of blades 725. The blade 725 can be configured to cancel at least some of forward waves 790. Since backward waves are generated largely based on the forward waves 790, cancellation of the forward waves 790 can reduce the backward waves (not shown). In the example of FIG. 7, the inner member 720 has a biconical shape. For instance, the blade 725 includes a first cone 725a and a second cone 725b. The first cone 725a can be referred to as an entry cone, and the second cone 725b can be referred to as an exit cone. The cones 725a, 725b may also be referred to as conical sections 725a, 725b. The forward waves 790a can enter through the entry cone 725a. In the entry cone 725a, the pressure wave component of the forward waves 790 is directed outside of the entry cone segment (indicated by arrows with reference number 790a). This pressure wave then returns and interferes with subsequent forward waves 790, effectively cancelling out. For instance, the deflected forward waves 790a return at an incident angle from the wall of the vessel and cancel at least some of the forward waves 790. Since backward waves are largely an artifact of forward waves 790, reducing the forward waves 790 would therefore reduce the backward waves. The entry cone 725a can be designed with an appropriate angle to facilitate cancellation of the forward waves 790.

Flow into the entry cone 725a may lead to energy loss due to the tapering of the entry cone 725a. For example, the tapering of the entry cone 725a increases resistance and decreases velocity. Accordingly, the blade 725 can include the exit cone 725b, which can accelerate blood flow velocity and compensate for the energy loss during the flow into the entry cone 725a. The exit cone 725b can be designed with an appropriate angle to facilitate acceleration of the forward waves 790 passing through the exit cone 725b. The exit cone 725b can have a different geometry (e.g., smaller taper) to accelerate velocity of the forward waves 790. The angle of the entry cone 725a and the angle of the exit cone 725b may be different. In some cases, the angle of the exit cone 725b is less than the angle of the entry cone 725a. A pressure drop may be created, for example, due to different angles, and flow velocity may be accelerated in the exit cone 725b. Accordingly, the entry cone 725a and the exit cone 725b can be asymmetrical or have different shapes. In some cases, the angle of the cone 725a, 725b may be referred to as a tapering angle. In some implementations, the blade 725 only includes an entry cone 725a for cancelling the forward waves 790. In such implementations, there may not be acceleration of the forward waves 790 after passing through the entry cone 725a.

The biconical shape is shown in FIG. 7 for illustrative purposes, and the blade 725 can have any shape or surface that can cancel at least a portion of the forward waves 790. The blade 725 can be designed with appropriate axial and/or cross-sectional angles to facilitate cancellation of forward waves 790. As described above, the blade 725 may be constructed of any suitable material, including a mesh or solid material such as PTFE or other synthetic materials previously established as safe for intravascular use. For example, the cones 725a, 725b may be made of a mesh or solid material, such as a polymer or biomaterial, a series of struts, blades, or any combination thereof. The wave cancellation device 700 can be placed in the main PA 18 and/or within branch PAs as described herein.

As described herein, the target position of the implant device 700 can be determined based on different factors, such as remodeling and/or stiffening of vessels. In addition, one or more blades 725 can be stacked. For instance, sets of blades 725 can be placed at multiple locations along the longitudinal axis of the inner member 720 (e.g., along the length of the vessel). Features relating to examples and implementations described with respect to FIG. 7, for example, associated with wave cancellation, can be independently implemented from features relating to examples and implementations described with respect to other figures of the present disclosure.

### Valved Devices

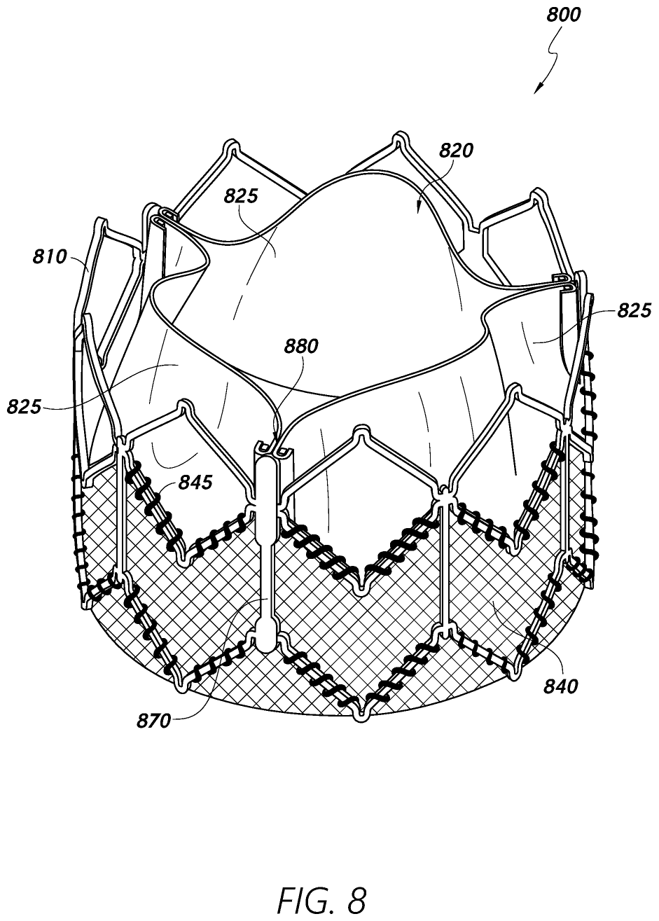

FIG. 8 is a view of an implant device 800 for modifying pulmonary waves in accordance with one or more examples. In some implementations, the implant device 800 can be a valved device, such as a prosthetic valve. In some cases, the valved device 800 can be located in distal pulmonary artery branches to prevent backward waves from reaching the main pulmonary artery during systole.

The valve 800 in the in the example of FIG. 8 generally can include a frame or stent 810, a leaflet structure 820 supported by the frame 810, and a skirt 840 secured to the outer surface of the leaflet structure 820. The valve 800 may be implanted in the annulus of a native valve of the heart or adapted to be implanted in in various other ducts or orifices of the body. The valve 800 and frame 810 can be configured to be radially collapsible to a collapsed or crimped state for introduction into the body on a delivery catheter and radially expandable to an expanded state for implanting the valve at a desired location in the body (e.g., a pulmonary artery). The frame 810 can be made of a plastically-expandable material that permits crimping of the valve 800 to a smaller profile for delivery and expansion of the valve 800 using an expansion device such as the balloon of a balloon catheter. Exemplary plastically-expandable materials that can be used to form the frame 810 may include stainless steel, a nickel-based alloy (e.g., a nickel-cobalt-chromium alloy), polymers, or combinations thereof. Alternatively, the valve 800 can be a self-expanding valve wherein the frame 810 is made of a self-expanding material such as Nitinol. A self-expanding valve can be crimped to a smaller profile and held in the crimped state with a restraining device such as a sheath covering the valve 800. When the valve 800 is positioned at or near the target site, the restraining device can be removed to allow the valve 800 to self-expand to its expanded, functional size.

The skirt 840 can be formed, for example, of polyethylene terephthalate (PET) ribbon. The skirt 840 can be secured to the inside of the frame 810 via Lenzing sutures 845. The leaflet structure 820 can be attached to the skirt 840 via a thin PET reinforcing strip, which enables a secure suturing and protects the pericardial tissue of the leaflet structure 820 from tears. The leaflet structure 820 can be sandwiched between the skirt 840 and the thin PET strip. The suture that secures the PET strip and the leaflet structure 820 to the skirt 840 can be any suitable suture, such as an Ethibond suture. The leaflet structure 820 can be formed of bovine pericardial tissue, biocompatible synthetic materials, or various other suitable natural or synthetic materials as known in the art.

The leaflets 825 can be secured to one another at their adjacent sides to form commissures 880 of the leaflet structure 820 (the edges where the leaflets come together). The leaflet structure 820 can be secured to frame 810 using suitable techniques and mechanisms. For example, the commissures 880 of the leaflet structure 820 desirably are aligned with the support posts 870 and secured thereto using sutures. The point of attachment of the leaflets to the posts 870 can be reinforced with bars, which desirably are made of a relatively rigid material (compared to the leaflets), such as stainless steel.

In the example of FIG. 8, the leaflet structure 820 includes three leaflets 825, which can be arranged to collapse in a tricuspid arrangement, as shown in FIG. 8. The leaflet structure 820 can have a mono-leaflet, bi-leaflet, tri-leaflet configuration, etc. as appropriate. The leaflets 825 can open and close to prevent backward waves from reaching the main pulmonary artery during systole. According to certain aspects, the frame 810 can be considered to be an outer member of the implant device 800, and the leaflet structure 820 and the leaflets 825 can be considered to be an inner member and one or more blades of the implant device 800, respectively.

As described herein, the target position of the implant device 800 can be determined based on different factors, such as remodeling and/or stiffening of vessels. In addition, one or more implant devices 800 can be stacked. For instance, the implant devices 800 can be placed at multiple locations along the length of one or more vessels. Features relating to examples and implementations described with respect to FIG. 8, for example, associated with valved devices, can be independently implemented from features relating to examples and implementations described with respect to other figures of the present disclosure.

### Increased Frictional Orifice/Linings

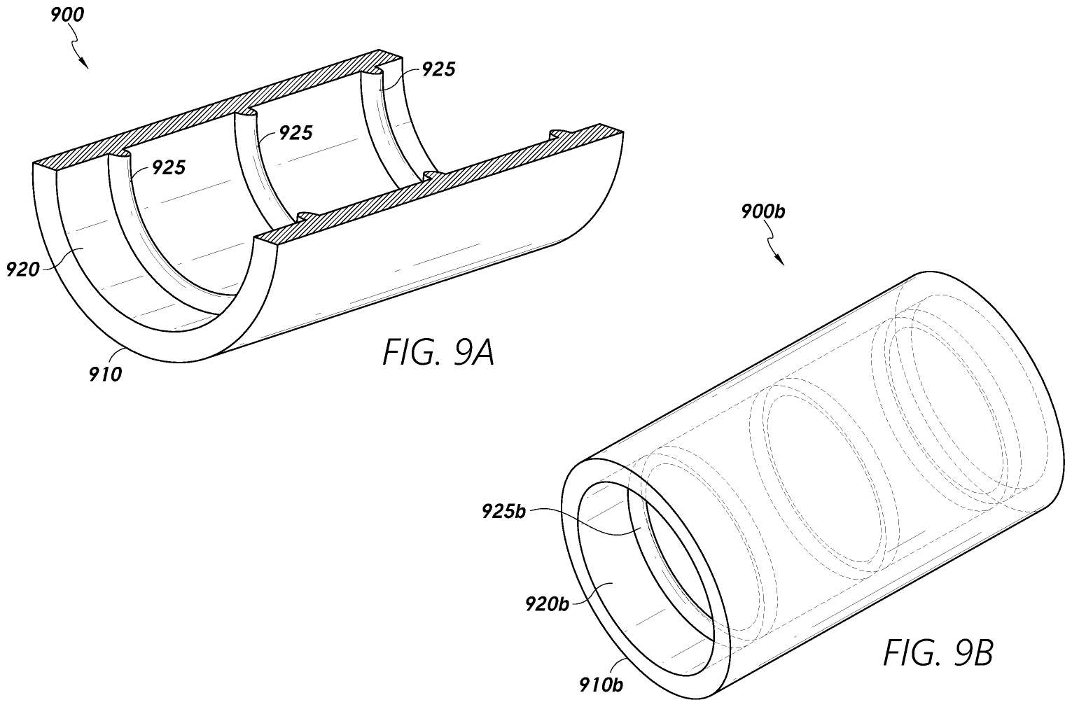

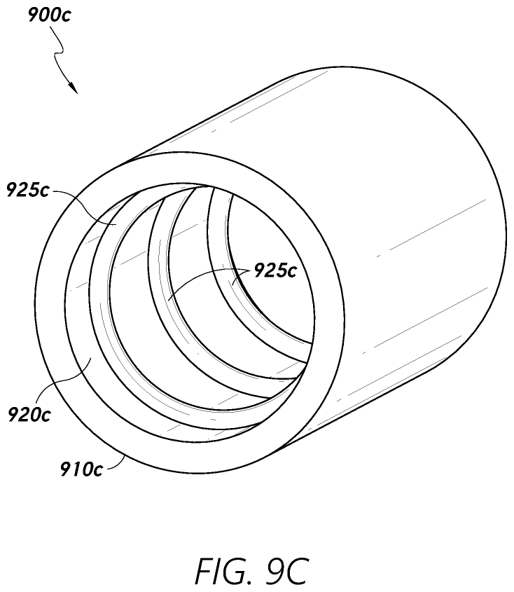

FIG. 9A is a perspective view of an implant device 900 for modifying pulmonary waves in accordance with one or more examples. For instance, the implant device 900 can include an outer member 910 and an inner member 920. In some examples, the outer member 910 and the inner member 920 can be integrated or formed as a single continuous structure or member. In other examples, the outer member 910 and the inner member 920 can be separate components. In some implementations, the implant device 900 is configured to provide increased friction, for example, along the wall of the inner member 920. For instance, the inner member 920 can include one or more ribbed linings or portions 925. The ribbed linings 925 can accelerate flow in the center of the lumen in the main PA (or another pulmonary vessel) and provide slower or no flow along the walls of the inner member 920. The ribbed linings 925 can accelerate flow of forward waves. The flow dynamic can reduce the afterload associated with backward waves. Accordingly, the implant device 900 can reduce interference between the forward waves and backward waves. In the example of FIG. 9A, the implant device 900 is a partial cylinder (e.g., a half cylinder). However, the implant device 900 can have any shape that is suitable and can be a partial geometric solid having an appropriate shape. In other implementations, the implant device 900 can include rough spikes and/or studs in order to create increased friction along the wall of the inner member 920, for example, instead of or in addition to ribbed linings 925. Such spiked and/or studded linings or portions can provide a roughened surface to accelerate flow in the center of the lumen of the main PA (or another pulmonary vessel).

FIG. 9B is a perspective view of an implant device 900b for modifying pulmonary waves in accordance with one or more examples. In some aspects, the implant device 900b can be similar to the implant device 900 in FIG. 9A, but the implant device 900b can be a full cylinder (or a full geometric solid having an appropriate shape). The implant device 900b can include an outer member 910b and an inner member 920b. In some examples, the outer member 910b and the inner member 920b can be integrated or formed as a single continuous structure or member. In other examples, the outer member 910b and the inner member 920b can be separate components. In some implementations, the implant device 900b is configured to provide increased friction, for example, along the wall of the inner member 920b. For instance, the inner member 920b can include one or more ribbed linings or portions 925b. The ribbed linings 925b can accelerate flow in the center of the lumen in the main PA (or another pulmonary vessel) and provide slower or no flow along the walls of the inner member 920b. The ribbed linings 925b can accelerate flow of forward waves. The flow dynamic can reduce the afterload associated with backward waves. Accordingly, the implant device 900b can reduce interference between the forward waves and backward waves. In other implementations, the implant device 900b can include rough spikes and/or studs in order to create increased friction along the wall of the inner member 920b, for example, instead of or in addition to ribbed linings 925b. Such spiked and/or studded linings or portions can provide a roughened surface to accelerate flow in the center of the lumen of the main PA (or another pulmonary vessel).

FIG. 9C is a perspective view of an implant device 900c for modifying pulmonary waves in accordance with one or more examples. In some aspects, the implant device 900c can be similar to the implant device 900b in FIG. 9B. The implant device 900c can include an outer member 910c and an inner member 920c. In some examples, the outer member 910c and the inner member 920c can be integrated or formed as a single continuous structure or member. In other examples, the outer member 910c and the inner member 920c can be separate components. In some implementations, the implant device 900c is configured to provide increased friction, for example, along the wall of the inner member 920c. For instance, the inner member 920c can include one or more ribbed linings or portions 925c that can accelerate flow in the center of the lumen in the main PA (or another pulmonary vessel) and provide slower or no flow along the walls of the inner member 920c. The ribbed linings 925c can accelerate flow of forward waves. As described in connection with FIG. 9B, the flow dynamic can reduce the afterload associated with backward waves, and the implant device 900c can reduce interference between the forward waves and backward waves. The implant device 900c may also include rough spikes and/or studs in order to create increased friction along the wall of the inner member 920b, for example, instead of or in addition to ribbed linings 925c. According to certain aspects, the ribbed linings 925, 925b, 925c and/or spiked and/or studded linings can be considered to be one or more blades of the implant device 900, 900b, 900c.

As described herein, the target position of the implant device 900, 900b, 900c can be determined based on different factors, such as remodeling and/or stiffening of vessels. In addition, one or more implant devices 900, 900b, 900c can be stacked. For instance, the implant devices 900, 900b, 900c can be placed at multiple locations along the length of one or more vessels. The implant device 900, 900b, 900c can be constructed of any suitable material, including the examples of materials described herein. Features relating to examples and implementations described with respect to FIGS. 9A-9C, for example, associated with increased friction, can be independently implemented from features relating to examples and implementations described with respect to other figures of the present disclosure.

### Increased Speed at Center

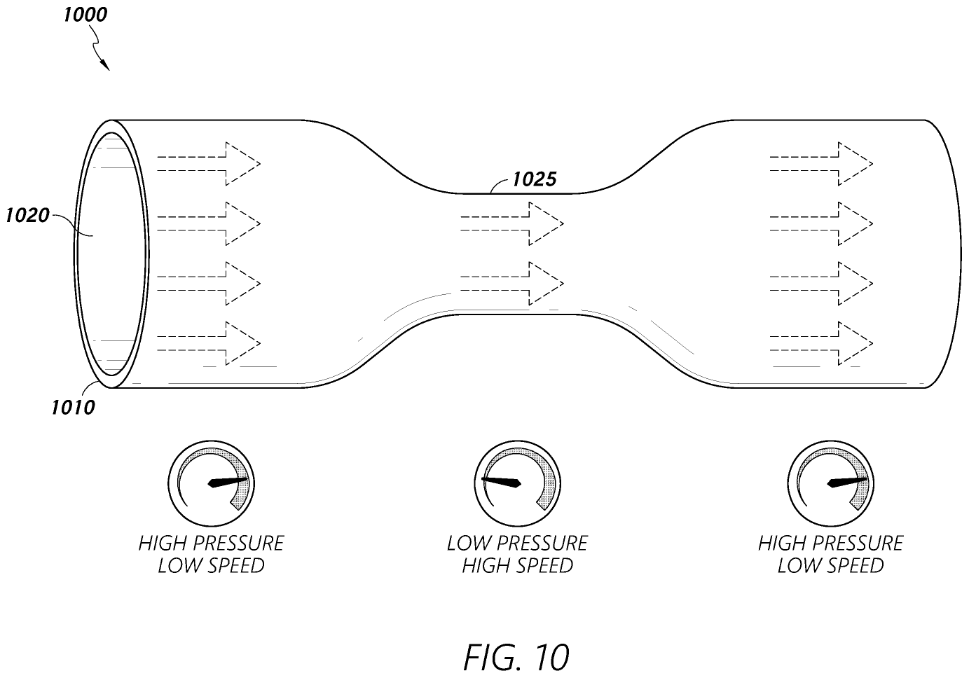

FIG. 10 is a view of an implant device 1000 for modifying pulmonary waves in accordance with one or more examples. For instance, the implant device 1000 can include an outer member 1010 and an inner member 1020. In some examples, the outer member 1010 and the inner member 1020 can be integrated or formed as a single continuous structure or member. In other examples, the outer member 1010 and the inner member 1020 can be separate components. In some implementations, the implant device 1000 is configured to accelerate flow in the center of the inner member 1020. For example, the inner member 1020 can include a constriction 1025 that decreases the pressure of the flow and increase the speed of the flow. In some cases, the constriction 1025 may also be referred to as a Venturi. The constriction 1025 can be placed in the main PA or another pulmonary vessel. The constriction 1025 can accelerate flow in the center of the lumen in the main PA or another pulmonary vessel. As shown in FIG. 10, flow can enter the implant device 1000 at a first end, and the pressure of the flow can be high and the speed of the flow can be low at the first end. The flow can proceed through the constriction 1025, and the pressure of the flow can be lower than the pressure at the first end and the speed of the flow can be higher than the speed at the first end. The flow can exit the implant device 1000 at a second end, and the pressure of the flow can be high and the speed of the flow can be low at the second end compared to the pressure and speed through the constriction 1025. For instance, the pressure and speed of the flow at the second end can be similar to the pressure and the speed of the flow at the first end. The constriction 1025 can accelerate flow of forward waves and reduce the RV afterload associated with backward waves. Accordingly, the implant device 1000 can reduce interference between the forward waves and backward waves. According to certain aspects, the constriction 1025 can be considered to be a blade of the implant device 1000.

As described herein, the target position of the implant device 1000 can be determined based on different factors, such as remodeling and/or stiffening of vessels. In addition, one or more implant devices 1000 can be stacked. For instance, the implant devices 1000 can be placed at multiple locations along the length of one or more vessels. The implant device 1000 can be constructed of any suitable material, including the examples of materials described herein. Features relating to examples and implementations described with respect to FIG. 10, for example, associated with increased speed at center, can be independently implemented from features relating to examples and implementations described with respect to other figures of the present disclosure.

### Choke Point for Asynchronous Flow

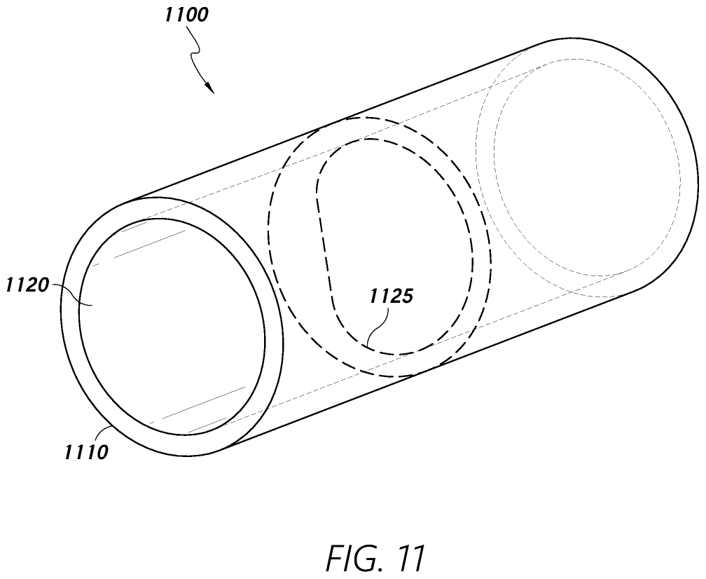

FIG. 11 is a perspective view of an implant device 1100 for modifying pulmonary waves in accordance with one or more examples. For instance, the implant device 1100 can include an outer member 1110 and an inner member 1120. In some examples, the outer member 1110 and the inner member 1120 can be integrated or formed as a single continuous structure or member. In other examples, the outer member 1110 and the inner member 1120 can be separate components. In some implementations, the implant device 1100 is configured to create asynchrony in timing of backward waves from the LPA and the RPA. The implant device 1100 may also be configured to accelerate flow in the center of the inner member 1120. For example, the inner member 1120 can include a choke point 1125 that has an asymmetrical shape such that backward waves from the LPA and the RPA can be asynchronous in timing. In the example of FIG. 11, the inner member 1120 can have a D-shaped choke point 1125, but the choke point 1125 can have any shape that is suitable for creating asynchrony for backward waves from the LPA and the RPA. The choke point 1125 can reduce additive effect or summation of the backward waves from the LPA and the RPA. The choke point 1125 can also accelerate flow of forward waves in the middle of the inner member 1120 and reduce the RV afterload associated with backward waves. Accordingly, the implant device 1100 can reduce interference between the forward waves and backward waves. According to certain aspects, the choke point 1125 can be considered to be a blade of the implant device 1100.

As described herein, the target position of the implant device 1100 can be determined based on different factors, such as remodeling and/or stiffening of vessels. In addition, one or more implant devices 1100 can be stacked. For instance, the implant devices 1100 can be placed at multiple locations along the length of one or more vessels. The implant device 1100 can be constructed of any suitable material, including the examples of materials described herein. Features relating to examples and implementations described with respect to FIG. 11, for example, associated with a choke point for asymmetry or asynchronous flow, can be independently implemented from features relating to examples and implementations described with respect to other figures of the present disclosure.

### Implantation Procedures

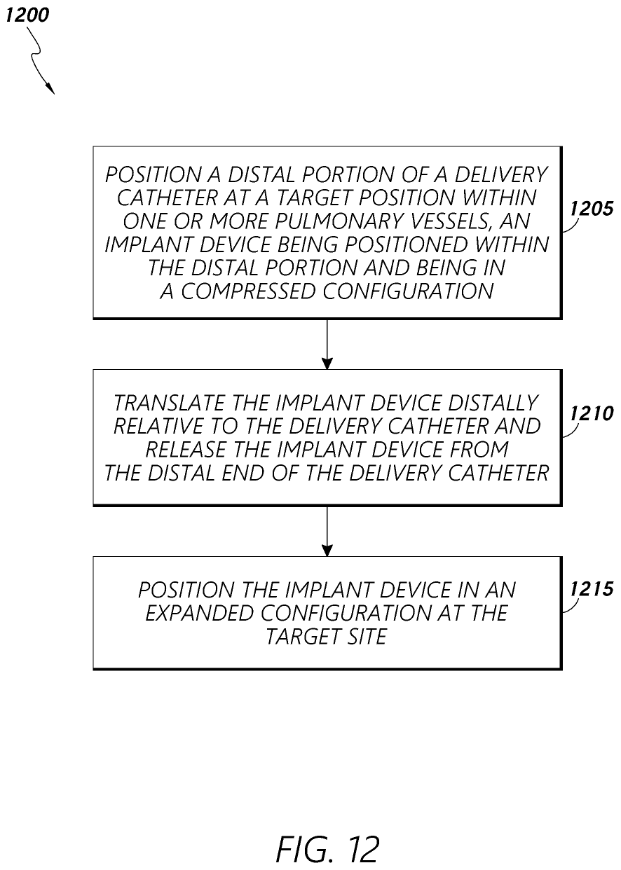

FIG. 12 provides a flow diagram illustrating a process 1200 for implanting an implant device for modifying pulmonary waves in accordance with one or more examples. For example, an implant device according to the present disclosure, such as implant devices described with respect to FIGS. 2-11 may be delivered and implanted within pulmonary arteries or other vessels of a patient. In some implementations, the implant device can include an outer member configured to position the implant device within one or more pulmonary vessels, and an inner member comprising one or more blades configured to modify one or more of forward waves or backward waves within the one or more pulmonary vessels to reduce interference between the forward waves and the backward waves during systole, where the one or more blades have a shape or surface to direct at least a portion of the forward waves to a region (e.g., a periphery) of the one or more pulmonary vessels. In some cases, the implant device may be in a compressed configuration or an expanded configuration. For instance, the implant device may be in a compressed configuration prior to releasing from a delivery catheter and may be changed to an expanded configuration for positioning at a target site. In the description of FIG. 12, the implant device 200 in FIG. 2A is used as an example implant device.

At block 1205, the process 1200 involves positioning a distal portion of a delivery catheter at a target position within one or more pulmonary vessels, an implant device 200 being positioned within the distal portion and being in a compressed configuration. At block 1210, the process 1200 involves translating the implant device 200 distally relative to the delivery catheter and releasing the implant device 200 from the distal end of the delivery catheter. At block 1215, the process 1200 involves positioning the implant device 200 in an expanded configuration at the target site. For example, the implant device 200 can assume the expanded configuration after the implant device 200 is released from the distal end of the delivery catheter.

According to certain aspects, standard delivery and implantation techniques and systems can be employed to implant the implant device 200. In some cases, implantation of the implant device 200 may be performed percutaneously, under fluoroscopy and echocardiographic guidance. Given the different planes, simultaneous transthoracic or transesophageal echocardiographic guidance may be utilized. The implant device 200 can be deployed consistent with standard clinical practice for delivering stented or valved devices.

In some implementations, a process for delivering the implant device 200 to the target site can include a minimally invasive transcatheter delivering technique. For example, the implant device 200 can be delivered to a target site via the superior vena cava (SVC) or via the inferior vena cava (IVC). In some implementations, a transjugular or trans-subclavian approach can be used. Alternatively, a transfemoral approach can be used to position the delivery system into the inferior vena cava.

Delivery systems may be used to position catheter tips and/or catheters to various areas of or near a human heart. It will be understood that the description can refer or generally apply to positioning of catheter tips and/or catheters from a first body chamber or lumen into a second body chamber or lumen, where the catheter tips and/or catheters may be bent when positioned from the first body chamber or lumen into the second body chamber or lumen. A body chamber or lumen can refer to any one of a number of fluid channels, blood vessels, and/or organ chambers (e.g., heart chambers). Additionally, reference herein to “catheters,” “tubes,” “sheaths,” “steerable sheaths,” and/or “steerable catheters” can refer or apply generally to any type of elongate tubular delivery device comprising an inner lumen configured to slidably receive instrumentation, including for example delivery catheters and/or cannulas.

### Additional Description of Examples

Provided below is a list of examples, each of which may include aspects of any of the other examples disclosed herein. Furthermore, aspects of any example described above may be implemented in any of the numbered examples provided below.

Example 1: An implant device comprising: an outer member configured to position the implant device within one or more pulmonary vessels; and an inner member comprising one or more blades configured to modify one or more of forward waves or backward waves within the one or more pulmonary vessels to reduce interference between the forward waves and the backward waves during systole, the one or more blades having a shape or surface that directs a portion of the forward waves to a region of the one or more pulmonary vessels.

Example 2: The implant device of any example herein, in particular example 1, wherein the one or more blades are configured to divert the forward waves to a region of the one or more pulmonary vessels.

Example 3: The implant device of any example herein, in particular examples 1-2, wherein the one or more blades flare outward to divert the forward waves.

Example 4: The implant device of any example herein, in particular example 1, wherein the one or more blades include a curved blade configured to converge the backward waves.

Example 5: The implant device of any example herein, in particular example 4, wherein the curved blade includes a plurality of fenestrations to allow at least some of the forward waves to pass through.

Example 6: The implant device of any example herein, in particular examples 4-5, wherein the curved blade diverts at least some of the forward waves to a region of the one or more pulmonary vessels.

Example 7: The implant device of any example herein, in particular examples 1, wherein the one or more blades have a helical configuration to create a helical flow.

Example 8: The implant device of any example herein, in particular example 7, wherein the helical configuration includes a predetermined number of rotations for a diameter length of the inner member.

Example 9: The implant device of any example herein, in particular examples 7-8, wherein the one or more blades have a linear shape at a cross section across a diameter of the inner member.

Example 10: The implant device of any example herein, in particular example 1, wherein the one or more blades include one or more streamlined convex surfaces to accelerate a velocity of the forward waves.

Example 11: The implant device of any example herein, in particular example 10, wherein the one or more blades are configured to promote a Bernoulli effect on pulmonary pressure and velocity.

Example 12: The implant device of any example herein, in particular examples 10-11, wherein the velocity of the forward waves is higher at a distal end than the velocity of the forward waves at a proximal end.

Example 13: The implant device of any example herein, in particular examples 10-12, wherein the one or more blades have a biconvex shape.

Example 14: The implant device of any example herein, in particular example 1, wherein the one or more blades include a first conical section configured to direct the forward waves to a periphery of the one or more pulmonary vessels, and forward waves returned from the periphery cancel out a portion of the forward waves.

Example 15: The implant device of any example herein, in particular example 14, wherein the one or more blades include a second conical section configured to accelerate a velocity of the forward waves.

Example 16: The implant device of any example herein, in particular example 15, wherein a tapering angle of the first conical section is different from a tapering angle of the second conical section.

Example 17: The implant device of any example herein, in particular examples 14-16, wherein the cancellation of the portion of the forward waves reduces the backward waves.

Example 18: The implant device of any example herein, in particular example 1, wherein the implant device is positioned within a main vessel.

Example 19: The implant device of any example herein, in particular example 1, wherein the implant device is positioned within a branch vessel.

Example 20: The implant device of any example herein, in particular example 1, wherein the implant device is positioned within a main vessel and one or more branch vessels.

Example 21: The implant device of any example herein, in particular example 1, wherein a portion of the implant device in one branch vessel and a portion of the implant device within another branch vessel are asymmetrical.

Example 22: The implant device of any example herein, in particular example 1, wherein the one or more blades are positioned at a plurality of locations within the one or more pulmonary vessels.

Example 23: The implant device of any example herein, in particular example 1, wherein the outer member and the inner member are formed as a single continuous tube.

Example 24: The implant device of any example herein, in particular example 1, wherein the reduction of the interference between the forward waves and the backward waves reduces pulmonary pressure and right ventricular afterload.

Example 25: The implant device of any example herein, in particular example 1, wherein the implant device is a valved device positioned within a branch vessel and configured to prevent the backward waves from reaching a main vessel during systole.

Example 26: The implant device of any example herein, in particular example 1, wherein the one or more blades include a ribbed portion along a wall of the inner member that is configured to create friction.

Example 27: The implant device of any example herein, in particular example 26, wherein the ribbed portion is configured to accelerate flow of the forward waves through a center of the inner member.

Example 28: The implant device of any example herein, in particular example 1, wherein the one or more blades include a spiked portion or a studded portion along a wall of the inner member that is configured to create friction.

Example 29: The implant device of any example herein, in particular example 28, wherein the spiked portion or the studded portion is configured to accelerate flow of the forward waves through a center of the inner member.

Example 30: The implant device of any example herein, in particular example 1, wherein the one or more blades include a constriction along the inner member that is configured to accelerate flow of the forward waves.

Example 31: The implant device of any example herein, in particular example 1, wherein the one or more blades include an asymmetrical choke point that is configured to create asynchrony between the backward waves from a first branch pulmonary vessel and a second branch pulmonary vessel.

Example 32: The implant device of any example herein, in particular example 31, wherein the first branch pulmonary vessel is a left pulmonary artery and the second branch pulmonary vessel is a right pulmonary artery.

Example 33: The implant device of any example herein, in particular examples 31-32, wherein the asymmetrical choke point is configured to accelerate flow of the forward waves through a center of the inner member.

Example 34: A method of delivering an implant device, the method comprising: positioning a distal portion of a delivery catheter at a target position within a vessel, the implant device being in a compressed configuration and being within the distal portion of the delivery catheter, the implant device comprising an outer member configured to position the implant device within one or more pulmonary vessels, and an inner member comprising one or more blades configured to modify one or more of forward waves or backward waves within the one or more pulmonary vessels to reduce interference between the forward waves and the backward waves during systole, the one or more blades having a shape or surface that directs a portion of the forward waves to a region of the one or more pulmonary vessels; causing the implant device to assume an expanded configuration after releasing the implant device from the delivery catheter; and positioning the implant device in the expanded configuration at a target site.

Example 35: An implant device comprising: means for positioning the implant device within one or more pulmonary vessels; and means for modifying one or more of forward waves or backward waves within the one or more pulmonary vessels to reduce interference between the forward waves and the backward waves during systole, the means for modifying configured to direct a portion of the forward waves to a region of the one or more pulmonary vessels.

Example 36: The implant device of any example herein, in particular examples 1-35, wherein the implant device is sterilized.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. An implant device comprising:
an outer member configured to position the implant device within one or more pulmonary vessels; and
an inner member comprising one or more blades configured to modify one or more of forward waves or backward waves within the one or more pulmonary vessels to reduce interference between the forward waves and the backward waves during systole, the one or more blades having a shape or surface that directs a portion of the forward waves to a region of the one or more pulmonary vessels.

2. The implant device of claim 1, wherein the one or more blades are configured to divert the forward waves to a region of the one or more pulmonary vessels.

3. The implant device of claim 2, wherein the one or more blades flare outward to divert the forward waves.

4. The implant device of claim 1, wherein the one or more blades include a curved blade configured to converge the backward waves.

5. The implant device of claim 4, wherein the curved blade includes a plurality of fenestrations to allow at least some of the forward waves to pass through.

6. The implant device of claim 4, wherein the curved blade diverts at least some of the forward waves to a region of the one or more pulmonary vessels.

7. The implant device of claim 1, wherein the one or more blades have a helical configuration to create a helical flow.

8. The implant device of claim 7, wherein the helical configuration includes a predetermined number of rotations for a diameter length of the inner member.

9. The implant device of claim 1, wherein the one or more blades include one or more streamlined convex surfaces to accelerate a velocity of the forward waves.

10. The implant device of claim 9, wherein the one or more blades are configured to promote a Bernoulli effect on pulmonary pressure and velocity.

11. The implant device of claim 9, wherein the velocity of the forward waves is higher at a distal end than the velocity of the forward waves at a proximal end.

12. The implant device of claim 9, wherein the one or more blades have a biconvex shape.

13. The implant device of claim 1, wherein the one or more blades include a first conical section configured to direct the forward waves to a periphery of the one or more pulmonary vessels, and forward waves returned from the periphery cancel out a portion of the forward waves.

14. The implant device of claim 13, wherein the one or more blades include a second conical section configured to accelerate a velocity of the forward waves.

15. The implant device of claim 14, wherein a tapering angle of the first conical section is different from a tapering angle of the second conical section.

16. The implant device of claim 15, wherein the cancellation of the portion of the forward waves reduces the backward waves.

17. The implant device of claim 1, wherein the implant device is a valved device positioned within a branch vessel and configured to prevent the backward waves from reaching a main vessel during systole.

18. The implant device of claim 1, wherein the one or more blades include a ribbed portion along a wall of the inner member that is configured to create friction.

19. The implant device of claim 1, wherein the one or more blades include a constriction along the inner member that is configured to accelerate flow of the forward waves.

20. The implant device of claim 1, wherein the one or more blades include an asymmetrical choke point that is configured to create asynchrony between the backward waves from a first branch pulmonary vessel and a second branch pulmonary vessel.
